(19) United States
(12) Patent Application Publication
(10) Pub. No.: US 2018/0357382 A1
(43) Pub. Date: Dec. 13, 2018

(54) Inmate Bed Location System

(71) Applicant: Global Tel*Link Corporation, Reston, VA (US)

(72) Inventors: Stephen L. HODGE, Aubrey, TX (US); Anthony BAMBOCCI, Altoona, PA (US)

(21) Appl. No.: 15/618,367

(22) Filed: Jun. 9, 2017

(51) Int. Cl. G06F 19/00; G06Q 10/10; G06Q 10/08

(52) U.S. Cl. CPC G06F 19/327; G06Q 10/087; G06Q 10/1093

(57) ABSTRACT

An inmate bed location system and a method for operating the inmate bed location system is disclosed. The inmate bed location system includes at least one correctional facility that includes a bed tracking function, a central server that stores and collects bed availability information from the at least one correctional facility, and at least one of a correction agency and an inmate transportation agency. The central server coordinates with the at least one correctional facility to detect, in real-time or on a predetermined schedule, availability of beds within the at least one correctional facility and generate a schedule of availability of beds. The central server also receives requests from the correction agency requesting a bed for an inmate and coordinates the selection of an appropriate bed based on the schedule of availability and at least a bed profile associated with the bed and an inmate profile associated with the inmate.

### BACKGROUND

### Field

This disclosure relates to dynamic detection of bed availability within a controlled environment and generating bed itineraries that combine requests from transportation agencies and corrections agencies based on the detected bed availabilities within the controlled environment.

### Background

Many correction agencies have a need to find available short-term bed space for inmates. Different inmates have different criteria that are required for incarceration such as security requirements and correction agencies must locate correctional facilities that not only have available beds but that can also meet the security requirements or other criteria associated with the particular inmate. Currently, correction agencies can only individually contact various correctional facilities and request if there are beds available for rent or short-term use by the correction agency and that meet the criteria associated with the inmate. Time is wasted calling individual correctional facilities and to ensure that the security criteria associated with an inmate can he met by the correctional facility which is necessary to ensure the safety of the inmates and the public.

### BRIEF DESCRIPTION OF THE DRAWINGS/FIGURES

The accompanying drawings, which are incorporated herein and form a part of the specification, illustrate embodiments of the present disclosure and, together with the description, further serve to explain the principles of the disclosure and to enable a person skilled in the pertinent art to make and use the embodiments.

FIG. 1 illustrates a block diagram of an exemplary inmate bed location system, according to embodiments of the present disclosure.

FIG. 2 illustrates an exemplary central server for use in the exemplary inmate bed location system of FIG. 1, according to embodiments of the present disclosure.

FIG. 3 illustrates a block diagram of an exemplary bed inventory tracking system for use in the exemplary inmate bed location system of FIG. 1, according to embodiments of the present disclosure.

FIG. 4 illustrates a flowchart diagram of an exemplary method for generating a bed inventory update for use with the exemplary bed inventory tracking system of FIG. 3, according to embodiments of the present disclosure.

FIG. 5 illustrates a flowchart diagram of an exemplary method for generating bed candidates in the exemplary inmate bed location system of FIG. 1, according to embodiments of the present disclosure.

FIG. 6 illustrates a flowchart diagram of an exemplary method for filtering bed candidates by the exemplary central server of FIG. 2, according to embodiments of the present disclosure.

FIG. 7 illustrates a flowchart diagram of an exemplary method for generating a transportation itinerary in the exemplary inmate bed location system of FIG. according to embodiments of the present disclosure.

FIG. 8 illustrates a block diagram of a general purpose computer that may be used to perform various aspects of the present disclosure.

The present disclosure will be described with reference to the accompanying drawings. In the drawings, like reference numbers indicate identical or functionally similar elements. Additionally, the left most digit(s) of a reference number identifies the drawing in which the reference number first appears.

### DETAILED DESCRIPTION

The highly inefficient method of the prior art requires numerous calls and does not provide any guarantees that the correction agency will be able to locate available bed space for its inmates. Accordingly, there is a need to provide a centralized system for providing updated bed availability information and connecting appropriate correctional facilities to correction agencies based on the criteria of the particular inmate. Moreover, the centralized system should also be able to connect correctional facilities and correction agencies to transportation agencies to coordinate the transport of inmates. What is needed is a system that can connect disparate networks of correctional facilities to the needs of correction agencies in an efficient manner.

The following Detailed Description refers to accompanying drawings to illustrate exemplary embodiments consistent with the disclosure. References in the Detailed Description to “one exemplary embodiment,” “an exemplary embodiment,” “an example exemplary embodiment,” etc., indicate that the exemplary embodiment described may include a particular feature, structure, or characteristic, but every exemplary embodiment may not necessarily include the particular feature, structure, or characteristic. Moreover, such phrases are not necessarily referring to the same exemplary embodiment. Further, when a particular feature, structure, or characteristic is described in connection with an exemplary embodiment, it is within the knowledge of those skilled in the relevant art(s) to affect such feature, structure, or characteristic in connection with other exemplary embodiments whether or not explicitly described.

The exemplary embodiments described herein are provided for illustrative purposes, and are not limiting. Other exemplary embodiments are possible, and modifications may be made to the exemplary embodiments within the spirit and scope of the disclosure. Therefore, the Detailed Description is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (R(I)M); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc. Further, any of the implementation variations may be carried out by a general purpose computer, as described below.

For purposes of this discussion, any reference to the term “module” shall be understood to include at least one of software, firmware, and hardware (such as one or more circuit, microchip, or device, or any combination thereof), and any combination thereof. in addition, it will be understood that each module may include one, or more than one, component within an actual device, and each component that forms a part of the described module may function either cooperatively or independently of any other component forming a part of the module. Conversely, multiple modules described herein may represent a single component within an actual device. Further, components within a module may be in a single device or distributed among multiple devices in a wired or wireless manner.

The following Detailed Description of the exemplary embodiments will so fully reveal the general nature of the disclosure that others can, by applying knowledge of those skilled in relevant art(s), readily modify and/or customize for various applications such exemplary embodiments, without undue experimentation, without departing from the spirit and scope of the disclosure. Therefore, such modifications are intended to be within the meaning and plurality of equivalents of the exemplary embodiments based upon the teaching and guidance presented herein. It is to be understood that the phraseology or terminology herein is for the purpose of description and not of limitation, such that the terminology or phraseology of the present specification is to be interpreted by those skilled in relevant art(s) in light of the teachings herein.

Exemplary Inmate Bed Location system

FIG, 1 illustrates a block diagram of an exemplary inmate bed location system 100, according to embodiments of the present disclosure. Inmate bed location system 100 can be implemented in a controlled environment, such as a correctional facility, where occupancy of beds within the controlled environment are tracked. For example, in a correctional facility, a server can be used to track the availability of beds based on an associate between inmates within the correctional facility and the beds within the cell blocks of the correctional facility.

In some embodiments, inmate bed location system 100 includes a set of correctional facilities 110 which can include correctional facility 110A. and correctional facility 110B, central server 120, correction agency 130, and transportation agency 140. Although two correctional facilities, one correction agency, and one transportation agency is shown for simplicity, it is understood that inmate bed location system 100 can include any number of correctional facilities, correctional agencies, and transportation agencies. Correctional facility can be any facility that requires tracking associations between inmates and beds. Correctional agencies can be an agency that is responsible for managing correctional facilities and the inmates that are incarcerated within such facilities such as the Corrections Corporation of America (CCA) or law enforcement agencies such as the United States Immigration and Customs Enforcement (ICE) agency. Transportation agencies can be an agency such as Transcor America, LLC that is responsible for transporting inmates and detainees between correctional facilities which can generally be at the request of the correction agency.

Correctional facilities 110 are connected to central server 120 through a network 150. Correction agency 130 is connected to central server 120 through network 160. Transportation agency 140 is connected to central server 120 through network 160. Central server 120 acts as an intermediary between the set of correctional facilities 110, correction agency 130, and transportation agency 140 by acting as a hub for tracking bed inventory at correctional facilities 110, maintaining a database of bed availability at correctional facilities 110, receiving and processing requests from correction agency 130 for available beds, and generating transport itineraries in cooperation with transportation agency 140.

Networks 150, 160, and 170 may include any or all of a Local-Area Network (LAN), a Wide-Area Network (WAN), or the Internet, depending on the location of central server 120 in relation to correctional facilities 110, correction agency 130, and transportation agency 140. For example, networks 150, 160, and 170 can be implemented as a LAN when central server 120 is co-located with any of correctional facility 110A, correctional facility 110B, correction agency 130, and transportation agency 140. In another example, networks 150, 160, and 170 can be implemented as a WAN or the Internet when central server 120 is located remotely from correctional facility 110A, correctional facility 110B, correction agency 130, and transportation agency 140, In some embodiments, central server 120 is cloud-based and includes one or more cloud-based servers. In some embodiments, central server 120 is a server within a data center that supports the devices within correctional facilities 110, correction agency 130, and/or transportation agency 140.

In some embodiments, correctional facility 11.0A includes cell block 111, cell block 112, a mobile bed tracking device 113, a local server 114, and a workstation 115. The number of cell blocks and devices in correctional facilities 110 is merely exemplary. Cell blocks in a controlled environment, such as cell block 111 and cell block 112, generally can be divided into a certain number of cells each having a certain number of beds that are assigned to inmates of the controlled environment. For example, cell block 111 has two cells, cell 1 and cell 2, with cell 1 having four beds and cell 2 having three beds. It is understood. that the configuration of cell block 111 is merely exemplary and does not limit the number of cells and beds of this disclosure. Cell block 112 can have also be divided into two cells, each having two beds. Correctional facility 110B can be similarly configured with a certain number of beds and devices for tracking and transmitting the availability of the beds.

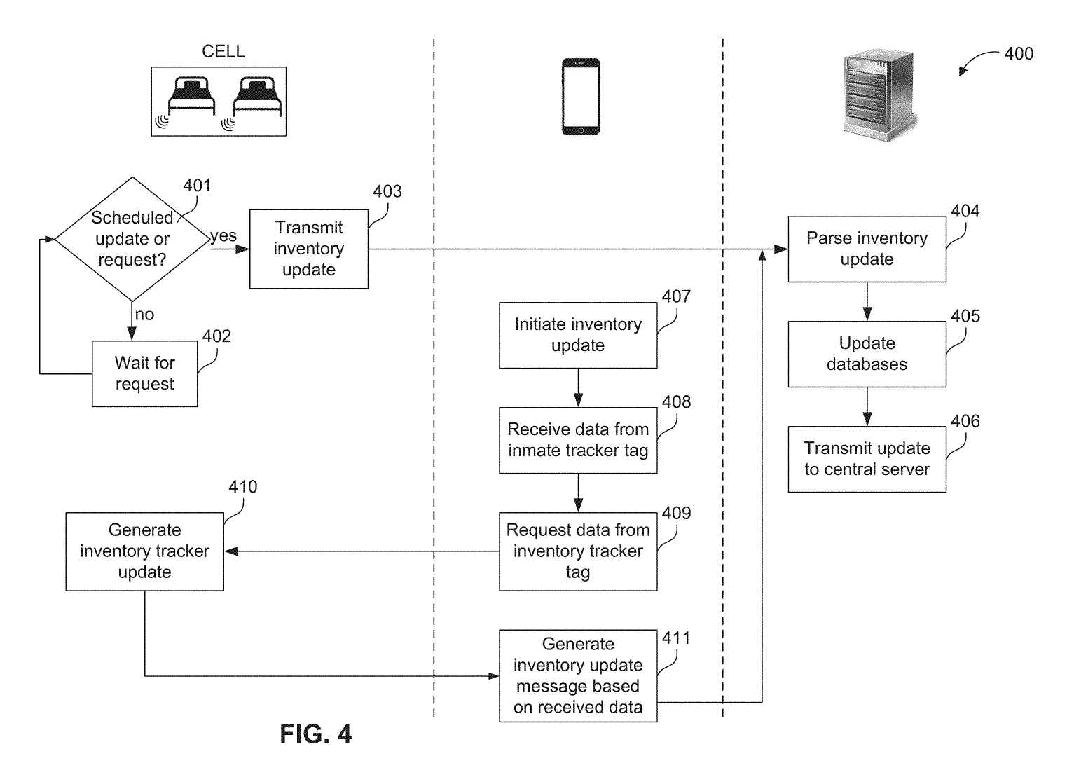

Correctional facility 110A maintains an inventory of the beds in cell block 111 and cell block 112 including tracking which beds have already been assigned to inmates and which beds are available (e.g., not assigned to an inmate). Correctional facilities 110 also maintains a calendar of for how long beds will be assigned to a particular inmate and for long the beds will be available (e.g., when an inmate is scheduled to arrive at correctional facility 110A). Correctional facilities 110 can employ automated method, a manual method, or a combination thereof for tracking the availability of its beds. In the automated method, beds deployed with bed inventory tracking devices automatically provide updates regarding availability of beds to local server 114. The updates are provided on a schedule or upon request from local server 114. In the manual method, personnel of correctional facility manually update the inventory using mobile bed tracking device 113 and/or workstation 115. These methods are discussed in further detail with regard to FIG. 4.

In some embodiments, central server 120 stores data provided by correctional facility 110A, correction agency 130, and transportation agency 140. Data from correctional facility 110A includes information about availability of beds and updates regarding the availability of the beds. The information includes inmate information regarding the inmate associated with each bed and dates on which the bed are or are not available (e.g., when a bed is associated with an inmate). Central server 120 includes one or more databases for storing information from correctional facilities 110 including correctional facility 110A and correctional facility 110B. Such information includes but is not limited to bed availability and inventory of correctional facilities 110, bed profiles for each of the beds in correctional facilities 110, inmate information of each of the inmates incarcerated in correctional facilities 110, and authentication information for the correctional facilities.

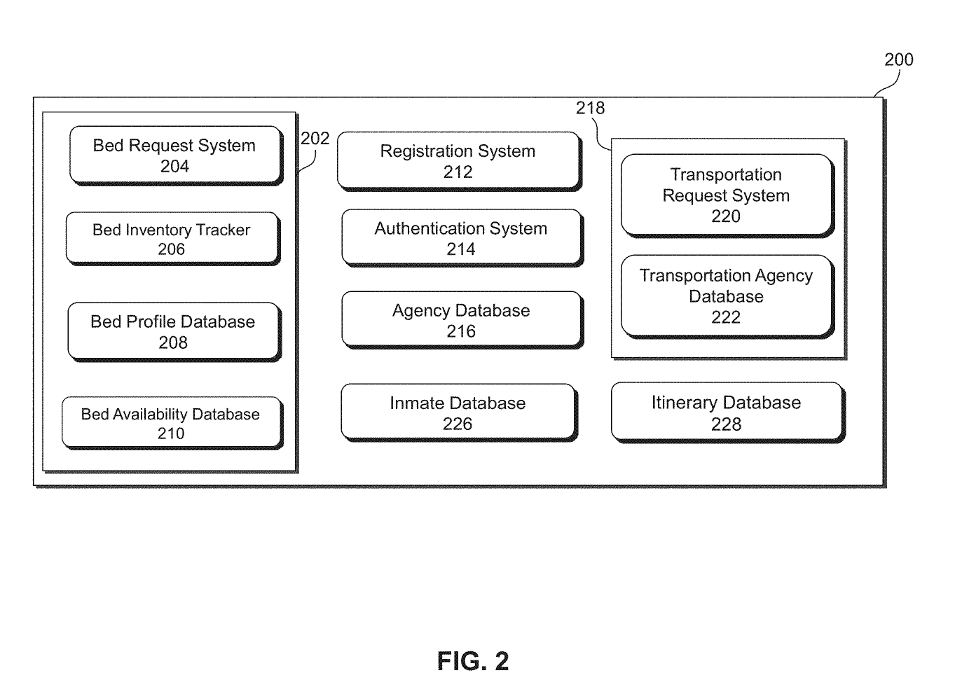

In some embodiments, central server 120 also includes data related to correction agency 130 and transportation agency 140. For example, central server 120 includes one or more databases for storing bed itineraries and transport itineraries. Bed itineraries are generated after correction agency 130 selects an available bed at a correctional facility within correctional facilities 110 for an inmate and include confirmed reservations for the inmate and the selected bed at the correctional facility. Bed itineraries include information regarding the selected correctional facility, the selected bed, the inmate, and the reservation details such as scheduled time period that the selected bed is needed. Transport itineraries are generated upon request or automatically after the generation of bed itineraries. Transport itineraries can include route information associated with two or more inmates that are being transported to a respective bed within a correctional facility or nearby correctional facilities. After generating a first bed itinerary, central server 120 identifies other previously generated bed itineraries that involve similar routes (e.g., another inmate is also scheduled to be transported to a bed in the same correctional facility). Based on parameters provided by correction agency 130 or transportation agency 140, central server 120identifies similarities between bed itineraries. As one non-limiting example, central server 120 compares a recently generated bed itinerary for an inmate scheduled to be associated with a bed in correctional facility 110B with another previously generated bed itinerary for another inmate who is scheduled to be associated with another bed in correctional facility 110B. Central server 120 identifies the similarities between the bed itineraries and notify correction agency 130 and/or transportation agency 140. In this manner, correction agency 130 and/or correctional facility 110B can save costs by having transportation agency 140 consolidate transportation of the inmates. Central server 120 coordinates transport plans with transportation agency 140 to create a transport itinerary that provides for the transportation of two or more inmates. Details regarding central server 120 are discussed further with respect to FIG. 2 below.

In some embodiments, correction agency 130 includes workstation 131 and mobile device 132. Workstation 131 accesses central server 120 through a website and mobile device 132 accesses central server 120 through a website or a mobile application installed on mobile device 132. Correction agency 130 such as ICE have a need to locate available bed space when transporting inmates and detainees. Correction agency 130 connects to central server 120 and transmits requests or receive push notifications of bed availability from correctional facilities 110.

In some embodiments, transportation agency 140 includes workstation 141 and mobile device 142. Workstation 141 accesses central server 120 through a website and mobile device 142 accesses central server 120 through a website or a mobile application installed on mobile device 142. Transportation agency 140 such as Transcor offers services to consolidate transportation of inmates to and from correctional facilities 110 based on the generated bed itineraries.

### Exemplary Central Server

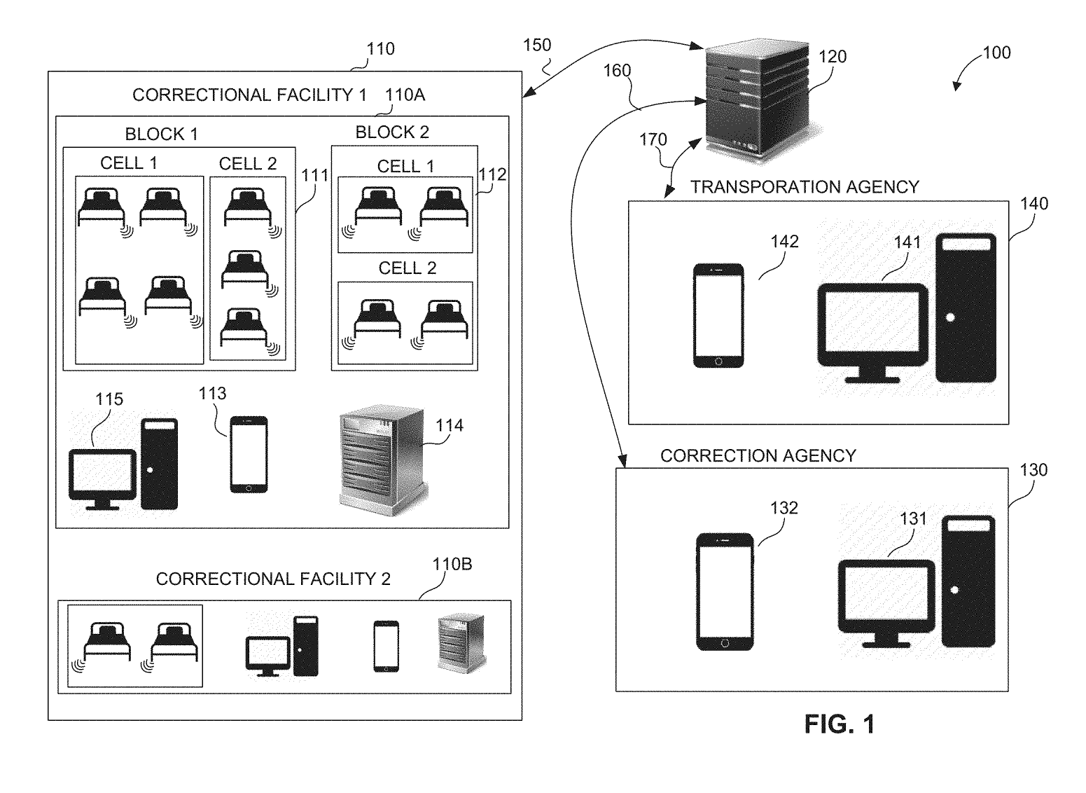

FIG. 2 illustrates a block diagram of central server 200, according to embodiments of the present disclosure. In an embodiment, central server 200 represents an exemplary embodiment of central server 120 of FIG. 1. Central server 200 includes but is not limited to bed processing subsystem 202, registration system 212, authentication system 214, agency database 216, transport processing system 218, inmate database 226, and itinerary database 228. Bed processing subsystem 202 includes one or more processors, computers, or servers identified as subsystems and are constructed as individual physical hardware devices, or as virtual devices, such as a virtual server. The number of processing subsystems can be scaled to match the number of simultaneous user connections desired to be supported by an inmate bed location system such as inmate bed location system 100 of FIG. 1. Processing subsystem 202 includes but is not limited to bed request system 204, bed inventory tracker 206, bed profile database 208, bed availability database 210.

In some embodiments, bed request system 204 controls the routing of communications related to requests for bed availabilities at correctional facilities within inmate bed location system 100 and reservations for beds. Requests and reservations are received from a correction agency that needs short-term or long-term housing for an inmate or inmates. Bed request system 204 coordinates with bed inventory tracker 206, bed profile database 208, bed availability database 210, and inmate database 226 to resolve requests for bed availabilities and reservations. For example, in response to a request, bed request system 204 parses the request for requirements for housing the inmate and determines available beds that meet the requirements of the inmate. In some embodiments, the request includes information that allows bed request system 204 to retrieve, from inmate database 226, an inmate profile associated with the inmate specified in the request, inmate profiles include but are not limited to security parameters associated with the flight risk, violence level, psychological assessment, gang affiliations, and known associates of the inmate specified in the request. Based on the inmate profile and any other criteria in the request, bed request system 204 identifies available beds that match the criteria associated with the inmate.

In some embodiments, bed inventory tracker 206 receives bed availability information from correctional facilities, such as correctional facility 110A and correctional facility 110B, associated with central server 200. Bed availability information includes information regarding the availability of beds within the correctional facility. Bed availability information are included as an automated notification from the correctional facility and/or transmitted in response to a request from bed inventory tracker 206. Bed inventory tracker 206 parses the availability information and updates an availability schedule stored in bed availability database 210. For example, correctional facility 110A transmits a message indicating that certain beds in cell block 111 have become available. Bed inventory tracker 206 receives the message and updates an availability schedule in bed availability database 210 to indicate that the beds indicated in the messages are now available for use by other inmates. An availability schedule tracks the availability of the beds made available by correctional facilities registered with central server 200.

Bed inventory tracker 206 also dynamically generates price points for beds based on the properties associated with the bed and of the correctional facility in which the bed is located. Bed inventory tracker 206 assigns different weights to properties and generate a price based on the weighted properties. For example, bed inventory tracker 206 determines that beds in maximum security facilities are priced higher than beds in lower security facilities and therefore have a greater weight. Other examples include facilities that have more amenities and facilities that have less bed density (e.g., less beds per cell). Accordingly, in some embodiments, beds that have properties with greater weights are assigned higher price points and vice versa.

After a correction agency confirms a reservation for a bed within a correctional facility, central server 200 generates a bed itinerary that includes information regarding the bed, the length of the stay, the inmate, and the correctional facility. As will be discussed later, bed itineraries are used to confirm a reservation and to generate transport itineraries for transporting the inmate to and from the correctional facility.

In some embodiments, bed profile database 208 stores bed profiles of all beds in correctional facilities connected to central server 200. Bed profiles include information regarding each of the beds that enable bed request system 204 to identify beds that match the criteria associated with an inmate in a request. Bed profiles include but are not limited to location of the bed such as the correctional facility, the specific block, and the specific cell in which the bed is located, security parameters of the bed such as whether the bed is located in a maximum security, medium security, or minimum security prison, and identities of any inmates located proximate to the bed. Bed profiles are provided by each correctional facility for each of their beds or are provided by administrative personnel associated with central server 200. In responding to requests for bed availabilities, bed request system 204 compares criteria associated with inmates in the request with parameters specified in bed profiles. Based on any matches between the criteria and the parameters of the bed profiles, bed request system 204 identifies any beds that meet the criteria. For example, an inmate may require a maximum security prison and has a known gang affiliation. Bed request system 204 searches through bed profile database 208 to locate any beds that are located in a maximum security prison and lack any inmates that have the same known gang affiliation. Bed request system 204 then determines availability of the identified beds based on information from bed availability database 210.

In some embodiments, bed profiles also include pricing information associated with the bed and that is established by the correctional facility in which the bed is located. Bed request system 204 utilizes the pricing information as part of the criteria for identifying beds that meet the request for available beds.

In some embodiments, registration system 212 includes components that allow correctional facilities, correctional agencies, and transportation agencies to register with central server 200. Registration with central server 200 can be a requirement to ensure security of transactions and protection of privacy of the inmates. Correctional facilities register using registration system 212. In some embodiments, central server 200 requires payment of a one-time or monthly fee to register with inmate bed location system 100. Once registered, a correctional facility submits bed information such as bed profiles and bed availability for storage in central server 200. Correctional agencies also register using registration system 212. In some embodiments, central server 200 requires payment by correctional agencies of an access fee such as a per-use or monthly fee to register with and access bed availability information in inmate bed location system 100. Once registered, a correction agency submits requests and make reservations for bed through central server 200. Transportation agencies also register using registration system 212. In some embodiments, central server 200 requires payment by transportation agencies of a one-time or monthly fee to register with inmate bed location system 100. Once registered, a transportation agency submits transportation information including transport routes, availability of transports, and a list of correctional facilities that are serviced by the transportation agency. Transportation agencies also accept transport itineraries generated for transporting inmates to and from certain correctional facilities.

In some embodiments, authentication system 214 performs authentication functions when a correctional facility, correction agency, or a transportation agency attempt to sign into inmate bed location system 100. Authentication system 214 receives authentication information associated with each correctional facility, correction agency, and transportation agency that attempts to sign in. Authentication information includes but is not limited to at least one of a username and password data, challenge questions, challenge answers, biometric data, device data such as make and model of a communication device, and/or location data. Biometric data includes one or more of a finger print, a hand print, a voice sample, an iris or retinal sample, a facial image of the user (2D or 3D), a gait identification, a hand geometry, a signature identification, an infrared camera identification, or any other biometric as deemed appropriate.

Authentication system 214 also determines whether generated bed candidates require additional approval and/or authorization. For example, certain inmates or beds in certain facilities may be classified as having a higher alert level based on certain parameters associated with the inmate, such as but not limited to the inmate's violence level, flight risk, and gang affiliations, as well as certain parameters associated with the bed, such as but not limited to gang violence level of neighboring inmates, flight risks of neighboring inmates, and gang affiliations of neighboring inmates. Accordingly, in some embodiments, based on the alert level of the inmate or bed candidates, authentication system 214 can require generated bed candidates associated with the inmate to be approved prior to transmission to the requesting correction agency.

In some embodiments, agency database 216 includes information regarding all correctional agencies that have registered with inmate bed location system. In some embodiments, transport processing system 218 includes transportation request system 220 and transportation agency database 222. Transport processing system 218 enables central server to identify and coordinate with transportation agencies to generate transport itineraries for transporting inmates to and from correctional facilities based on the bed requests from correctional agencies. Transport itineraries are generated after generation of a bed itinerary (e.g., when a correction agency has confirmed a bed reservation with central server 200) and specify transportation details of the inmate to and from the correction agency. Transportation details include but is not limited to the dates, the pickup time, the delivery time, the pickup location, and the delivery location of the inmate. Transportation request system 220 allows central server 200 to communicate with transportation agencies to submit transport itineraries to transportation agencies which can use the transport itineraries to offer transport services for the itinerary. Transportation agency database 222 includes information regarding the transportation agencies including their transport routes, the correctional agencies which they service, and pricing information.

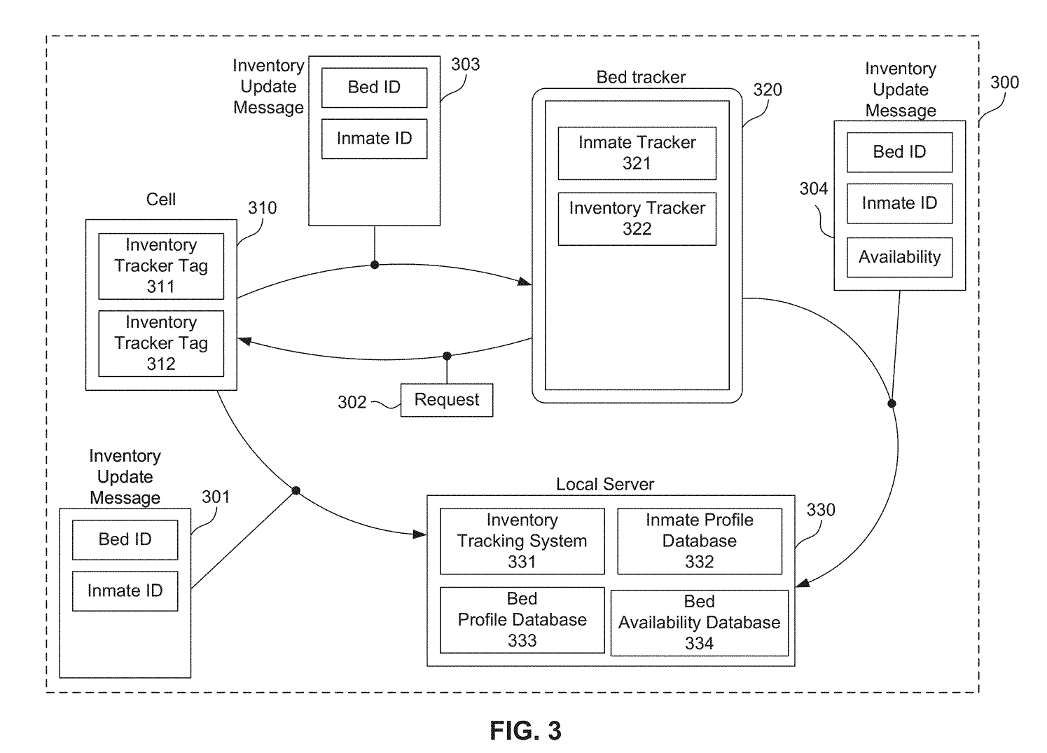

As discussed above, inmate database 226 includes inmate profiles associated with inmates detained within correctional facilities registered with central server 200. In some embodiments, inmate database 226 centrally stores information from an offender management system (OMS). In some embodiments, central server 200 is implemented as part of an OMS. Correctional facilities such as correctional facility 110A and correctional facility 110B provide automated or manual updates with OMS to provide information regarding inmates within each correctional facility for central storage within central server 200 and the OMS. Updates include any information about new or existing inmates within the correctional facility including but not limited to inmate counts (e.g., total number of inmates), new inmates, or inmates who have been released from the correctional facility. Automated updates occur on an automated basis such as on a schedule or at predetermined intervals. Manual updates occur on a manual basis such as on request by either administrators at the correctional facilities or at the OMS. In some embodiments, automated or manual updates include bed locator information regarding beds and associated inmates. Providing bed locator information from a correctional facility is further discussed with respect to FIG. 3. Itinerary database 228 includes bed itineraries and travel itineraries generated in response to the actions discussed above by components of central server 200.

Bed profile database 208, bed availability database 210, agency database 216, inmate database 226, transportation agency database 222, and itinerary database 228 can include any number of databases and/or servers, and stores and organizes data in a relational database. The databases run a database management system, such as MYSQL™, to provide an example.

### Exemplary Bed Inventory Update System

FIG. 3 illustrates a block diagram of an exemplary bed inventory update system 300 performed by a correctional facility for use in the exemplary inmate bed location system 100 of FIG. 1, according to embodiments of the present disclosure. Bed inventory update system 300 represents an exemplary embodiment performed by a correctional facility, such as correctional facility 110A. or correctional facility 110B, as described in FIG. 1, In some embodiments, bed inventory update system 300 includes cell 310, mobile bed tracking device 320, and local server 330. Cell 310 represents an exemplary embodiment of a cell within a correctional facility such as cell block 111 or cell block 112 as described in FIG. 1. Mobile bed tracking device 320 represents an exemplary embodiment of mobile bed tracking device 113 as described in FIG. 1. Local server 330 represents an exemplary embodiment of local server 114 as described in FIG. 1.

In some embodiments, cell 310 includes a bed with inventory tracker tag 311 and a bed with inventory tracker tag 312. Inventory tracker tag 311 and inventory tracker tag 312 are associated with a respective bed and include circuitry for identifying an inmate that is currently using the bed (e.g., through close-range detection of radio-frequency signals) and for providing bed availability updates to mobile bed tracking device 320 and local server 330. For example, inventory tracker tag 311 and inventory tracker tag 312 are configured to receive signals, such as radio-frequency (RF) signals, from a device associated with an inmate, such as a RF tag that transmits identification information of the inmate. Based on the received signals, inventory tracker tag 311 and inventory tracker tag 312 determines that the bed is not available because it is currently being utilized by the inmate associated with the received signal. Conversely, the absence of any signals received by inventory tracker tag 311 and inventory tracker tag 312 indicates that no inmate is currently using the bed and therefore the beds associated with inventory tracker tag 311 and inventory tracker tag 312 are available. In order to determine when beds are available, inventory tracker tag 311 and inventory tracker tag 312 is configured to detect inmate signals at predetermined times such as at midnight or later at night. The absence of an inmate signal at this time could be used to indicate the bed's availability. Inventory tracker tag 311 and inventory tracker tag 312 generate inventory update messages based on receiving or not receiving inmate signals.

In some embodiments, inventory tracker tag 311 and inventory tracker tag 312 transmit inventory update messages, such as inventory update message 301, directly to local server 330. Inventory update message 301 is transmitted at a predetermined time or upon request from local server 330. Inventory update message 301 includes any of a bed identifier identifying the bed associated with the inventory tracker tag, a facility identifier identifying a correctional facility in which the bed is located, a location identifier identifying a location (e.g., block, cell) of the bed within the correctional facility, and, if applicable, an inmate identifier determined from a signal received at the inventory tracker tag. Inventory update message 301 notifies local server 330 as to the availability of the bed associated with the bed identifier. If inventory update message 301 includes an inmate identifier, then local server 330 determines that the bed associated with the bed identifier is currently assigned to the inmate associated with the inmate identifier. In addition, in some embodiments, this determination is compared with an availability schedule of bed assignments that is manually updated by administrative personnel. In an embodiment, a comparison is used to verify that the inmate assigned to the bed is currently using the bed. If the inventory tracker tag does not receive a signal with an inmate identifier, the inventory update message would not include an inmate identifier or include an indication that the bed is currently available and not in use.

Local server 330 includes inventory tracking system 331, inmate profile database 332 and bed profile database 333. Inventory tracking system 331 receives inventory update message 301 and updates inmate profile database 332 and bed availability database 334 based on inventory update message 301. For example, inventory tracking system 331 updates inmate profile database 332 to indicate that the inmate associated with the inmate identifier is currently using the bed associated with the bed identifier in inventory update message 301. Inventory tracking system 313 further similarly updates bed availability database 334 to indicate that the bed associated with the bed identifier is currently in use by the inmate associated with the inmate identifier. As previously discussed, in some embodiments, inventory tracking system 331 includes a list of inmate-bed assignments stored in bed availability database 334 and that is manually updated by administrative personnel. Inventory tracking system 331 compares information in inventory update message 301 with the list of inmate-bed assignments to confirm that the appropriate inmate is currently using the bed associated with the bed identifier.

Inmate profile database 332 includes inmate profiles of all inmates incarcerated in bed inventory update system 300 of a correctional facility. As discussed above, inmate profiles contain information associated with inmates including security requirements (e.g., maximum security) and facility requirements associated with the inmate. Local server 330 further includes bed profile database 333 which includes bed profiles of all beds in bed inventory update system 300 of a correctional facility. As discussed above, bed profiles contain information about each bed including the correctional facility in which the bed is located and any inmates proximate to (e.g., in the same cell, in the same block) the bed. Local server 330 transmits updated bed profiles and inmate profiles to central server 200 at predetermined intervals and/or upon request by central server 200.

In some embodiments, inventory tracker tag 311 and inventory tracker tag 312 provides inventory update messages to mobile bed tracking device 320 which can be carried and used by guards of the controlled facility. Mobile bed tracking device 320 is implemented as any mobile device such as a smartphone, that allows guards of the controlled facility to manually request and update bed inventories as they walk around the controlled facility. For example, when mobile bed tracking device 320 is near inventory tracker tag 311 or inventory tracker tag 312, it submits a request 302 for an inventory update message. In response to receiving request 302, inventory tracker tag 311 or inventory tracker tag 312 responds by transmitting inventory update message 303 which has a similar format to inventory update message 301. Inventory update message 303 includes a bed identifier and, if applicable, an inmate identifier. Mobile bed tracking device 320 receives inventory update message 303.

In some embodiments, mobile bed tracking device 320 includes inmate tracker 321 and inventory tracker 322 which is implemented as systems for allowing users of mobile bed tracking device 320 to view and manually update information regarding the bed and inmate identified in inventory update message 303. Inmate tracker 321 allows the user to view information regarding the inmate identified by the inmate identifier of inventory update message 303. For example, inmate tracker 321 provides information regarding the inmate such as the expected bed or location of the inmate and therefore allows the user to confirm that the inmate is located in the proper bed. Inventory tracker 322 allows the user to view information regarding the bed identified by the bed identifier of inventory update message 303. For example, inventory tracker provides information regarding the bed such as the expected inmate that is assigned to the bed and therefore allows the user to confirm that the bed is being currently used by the correct inmate. Mobile bed tracking device 320 transmits an inventory update message 304 to local server 330 either upon request of a user of mobile bed tracking device 320, upon request of local server 330, or at predetermined intervals. Local server 330 processes inventory update message 304 in the manner described with respect to inventory update message 301.

Moreover, in some embodiments, inventory update message 304 further includes availability information provided by either inmate tracker 321 and/or inventory tracker 322. Availability information includes information manually input by user of mobile bed tracking device 320 such as expected availability schedule of the bed, and expected availability schedule of the inmate that is currently using the bed. Local server 330 updates bed availability database 334 based on the availability information provided by mobile bed tracking device 320.

### Exemplary Inmate Bed Location System Operation

Exemplary usage of inmate bed location system 100 and central server 200 in a controlled environment will be described with respect to FIGS. 4-7. The exemplary usage described in FIGS. 4-7 can be performed by processing logic that can comprise hardware (e.g., circuitry, dedicated logic, programmable logic, microcode, etc.), software (e.g., instructions executing on a processing device), or a combination thereof. For illustrative purposes, FIGS. 4-7 are described with respect to FIGS. 1-3 but are not limited to these example embodiments.

FIG. 4 illustrates a flowchart diagram of an exemplary method 400 for updating bed availability within a correctional facility in the exemplary inmate bed location system of FIG. 1, according to embodiments of the present disclosure. Method 400 can be performed by processing logic that can comprise hardware (e.g., circuitry, dedicated logic, programmable logic, microcode, etc.), software (e.g., instructions executing on a processing device), or a combination thereof. It is to be appreciated that additional steps, such as additional object recognition steps, may be performed. Moreover, not all steps may be needed to perform the disclosure provided herein. Further, some of the steps may be performed simultaneously, or in a different order than shown in FIG. 4, as will be understood by a person of ordinary skill in the art. Some steps can be combined with steps of methods from other figures and/or performed as a single step. Method 400 can be performed by bed inventory update system 300.

In 401, an inventory tracker tag associated with a bed within a cell of correctional facility determines whether it is scheduled to transmit an inventory update message to local server 330 or if it has received a request to transmit an inventory update message. For example, the inventory tracker tag determines, based on a scheduled-update parameter, whether it should transmit and update message to local server 330. If yes, the inventory tracker tag requests a signal from a nearby inmate (e.g., such as an RFID signal from an RFID tag permanently attached to the inmate). The inventory tracker tag generates an inventory update message as discussed above with respect to FIG. 3 and, in 403, transmits the inventor update message to local server 330. If the inventory tracker tag is not scheduled or has not received a request, it continues to wait at 402.

In 404, local server 330 receives the inventory update message and parses the message for information associated with the bed such as the bed identifier, facility identifier, location identifier, and if applicable, an inmate identifier. In 405, based on the information in the inventory update message, local server 330 updates its databases including inmate profile database 332, bed profile database 333, and bed availability database 334. For example, the inventory update message indicates that a bed is no longer available and is currently being used by an inmate. In 405, local server 330 updates the appropriate databases based on the inventory update message. For example, local server 330 will update the inmate's inmate profile in inmate profile database 332, the bed's bed profile in bed profile database 333, and the availability of the bed in bed availability database 334, in 406, local server 330 will transmit an inventory update message to central server 200 that provides updated bed availability information for beds within the correctional facility.

In 407, mobile bed tracking device 32.0 initiates an inventory update of a bed. For example, a user of mobile bed tracking device 320 may initiate the inventory update when checking out or checking in an inmate into the correctional facility. In some embodiments, mobile bed tracking device 320 detects a mobile update parameter that has been set by local server 330. Based on the detecting of the mobile update parameter, mobile bed tracking device 320 initiates an inventory update of 407. In 408, mobile bed tracking device 320 receives information from an inmate tracker tag. In some embodiments, the inmate tracker tag is attached to the inmate and/or the bed to which the inmate is assigned. The information includes but is not limited to an inmate identifier associated with the inmate, a facility identifier, a location identifier, and a bed identifier associated with the bed. In 409, mobile bed tracking device 320 submits a request for updated information from the inventory tracker tag associated with the bed. In 410, the inventory tracker tag generates updated availability information regarding the bed. For example, the inventory tracker tag generates a message indicating the bed identifier associated with the bed as well as a notification as to whether the inmate is currently assigned to the bed. In 411, mobile bed tracking device 320 receives the updated information and generates an inventory update message that includes the bed availability information and any details regarding the bed and the inmate, such as the bed identifier and inmate identifier, respectively.

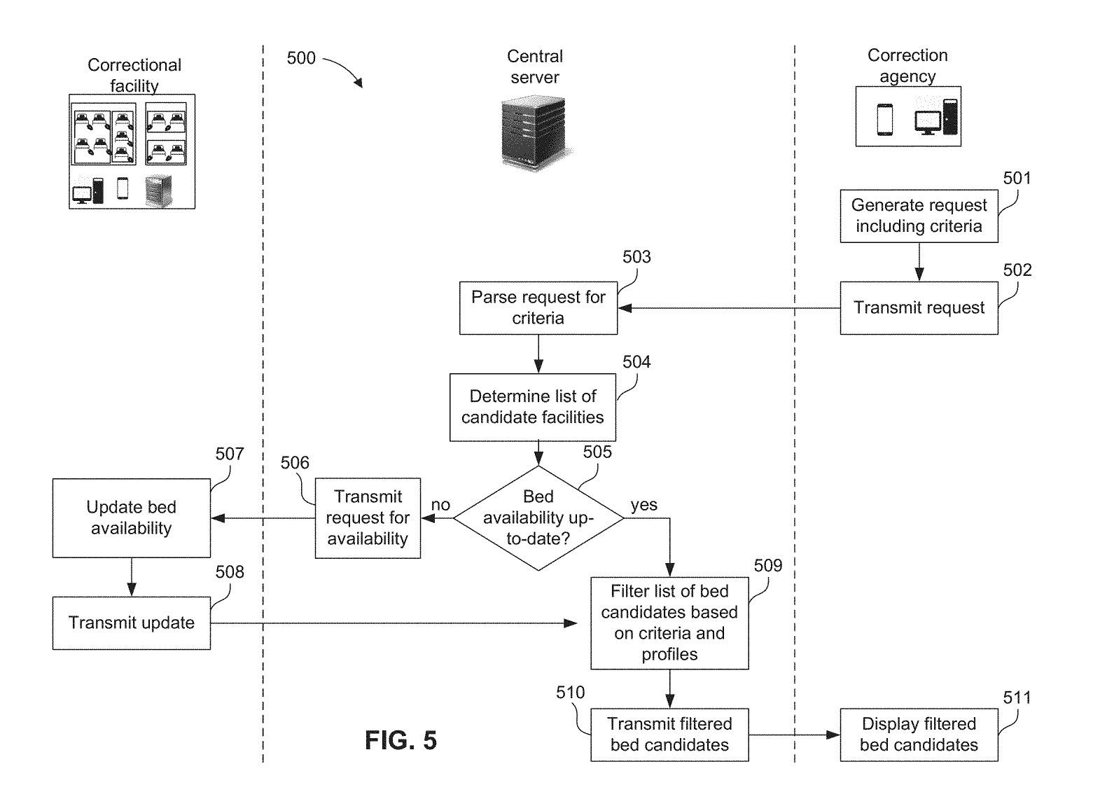

FIG. 5 illustrates a flowchart diagram of an exemplary method 500 for processing a bed availability request, according to embodiments of the present disclosure. Method 500 can be performed by processing logic that can comprise hardware (e.g., circuitry, dedicated logic, programmable logic, microcode, etc.), software (e.g., instructions executing on a processing device), or a combination thereof. It is to be appreciated that additional steps, such as additional object recognition steps, may be performed. Moreover, not all steps may be needed to perform the disclosure provided herein. Further, some of the steps may be performed simultaneously, or in a different order than shown in FIG. 5, as will be understood by a person of ordinary skill in the art. Some steps can be combined with steps of methods from other figures and/or performed as a single step. Method 500 can be performed by bed correctional facility HO, central server 200, and correction agency 130.

In 501, correction agency 130 generates a bed availability request for an inmate. The bed availability request includes information identifying the inmate (e.g., inmate identifier), location identifier, facility identifier, dates needed for the bed, and a length of time that the bed is needed. In some embodiments, additional criteria are included in the request such as the specific security requirements associated with the inmate, preferences such as a location (e.g., zip code) of the bed or distance from the inmate's current location (e.g., within 5 miles, within 10 miles), price limits, and facility amenities. In 502, correction agency 130 transmits the bed availability request to central server 200 for processing.

In 503, central server 200 receives and parses the request to determine the criteria for retrieving available beds. In 504, central server 200 identifies a list of facility candidates that meet the criteria associated with the facility within the bed availability request. For example, if the bed availability request includes facility criteria specifying a facility location, a distance from the inmate's current location, price limits, and/or facility amenities, central server 200 determines a list of facilities who best match the listed criteria. In 505, central server 200 begins to determine a list of available beds that are located within the candidate facilities. Central server 200 determines whether the bed information for the beds in the candidate facilities is up-to-date. For example, central server 200 may consider bed information stale if it has not been updated within a predetermined time period (e.g., within 4 hours, within 8 hours, within a day). If not, central server 200 transmits a bed update request to a correctional facility within the candidate facilities, For example, if central server 200 determines that bed availability information for correctional facility 110 is stale, central server 200 transmits a bed update request. The bed update request is a request for correctional facility to provide bed availability information to central server 200. In 507, correctional facility 110 receives the request from central server 200 and locally updates the bed availability information. In 508, correctional facility 110 transmits the updated bed availability information to central server 200.

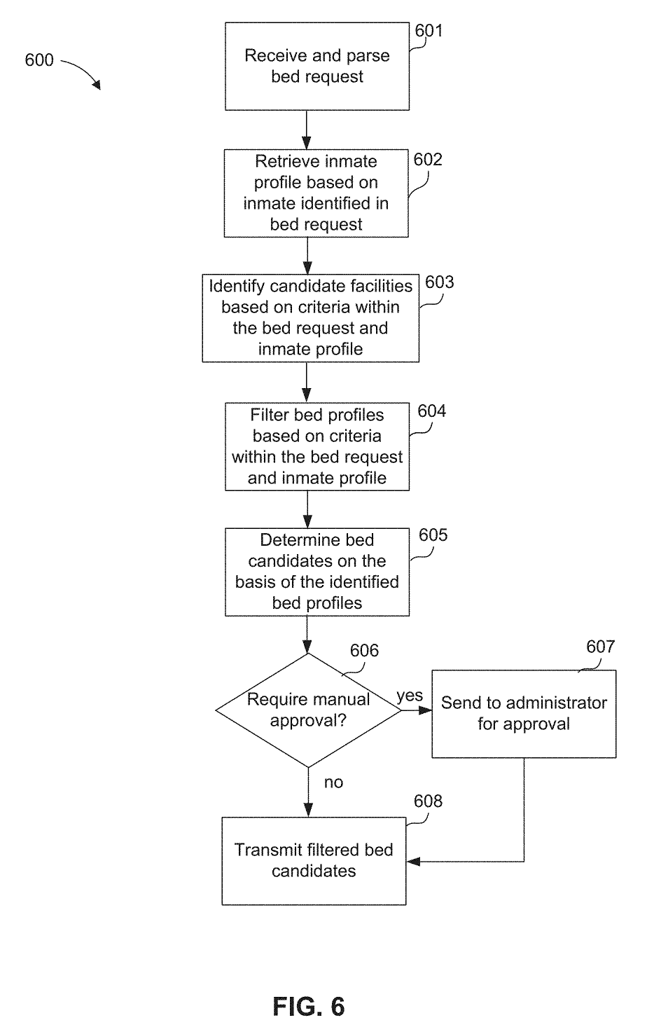

In 509, if central server 200 determines that the bed availability information for candidate facilities is up-to-date or after central server 200 receives updated bed availability information, central server 200 filters a list of bed candidates based on at least one of the availability information, the criteria in the bed availability request, bed profiles, and an inmate profile associated with the inmate identified in the bed availability request. Filtering process of central server 200 is discussed in further detail in FIG. 6. In 510, central server 200 transmits the filtered list of bed candidates to the requesting correction agency 130. In 511, correction agency 130 displays the filtered bed candidates for selection by a user at correction agency 130.

FIG. 6 illustrates a flowchart diagram of an exemplary method 600 for filtering bed candidates by central server 200 of FIG. 2, according to embodiments of the present disclosure. Method 600 can be performed by processing logic that can comprise hardware (e.g., circuitry, dedicated logic, programmable logic, microcode, etc.), software (e.g., instructions executing on a processing device), or a combination thereof, it is to be appreciated that additional steps, such as additional object recognition steps, may be performed. Moreover, not all steps may be needed to perform the disclosure provided herein. Further, some of the steps may be performed simultaneously, or in a different order than shown in FIG. 6, as will be understood by a person of ordinary skill in the art. Some steps can be combined with steps of methods from other figures and/or performed as a single step. Method 600 can be performed by central server 200.

In 601, central server 200 receives and parses a bed availability request from a correction agency, such as correction agency 130. In 602, central server 200 retrieves an inmate profile based on the inmate identified in the bed availability request. For example, the bed availability request includes an inmate identifier and central server 200 determines an identity of the inmate based on the inmate identifier. Based on the inmate identifier, central server 200 retrieves an inmate profile associated with the inmate identifier. In some embodiments, the inmate profile includes but is not limited to security parameters associated. with a flight risk of the inmate, violence level of the inmate, psychological assessment of the inmate, gang affiliations of the inmate, and known associates of the inmate. These security parameters specify the type of correctional facility that is appropriate for the inmate. For example, central server 200 eliminates certain bed candidates based on neighboring inmates who have the same gang affiliation as the inmate. As another example, central server 200 determines that beds in maximum security prisons are necessary for the inmate based on the flight risk or violence level of the inmate.

In 603, central server 200 identifies a list of candidate facilities based on the parameters within the bed availability request and the inmate profile. For example, the criteria within the bed availability request specifies economic restrictions associated with the request such as a price point and time restrictions such as the required dates and time period needed for the bed. As discussed above, the inmate profile includes restrictions based on the necessary amenities for incarcerating the inmate such as maximum or minimum security prisons. Central server 200 identifies the list of candidate facilities based on matching criteria of candidate facilities with the criteria specified in the bed availability request and the inmate profile. For example, central server 200 determines candidate facilities having beds available on the dates specified in the bed availability request, beds priced within the price limits specified in the bed availability request, and having amenities that meet the criteria of the inmate as specified in the inmate profile and/or the bed availability request.

With regard to pricing, in some embodiments, candidate facilities specifies specific price points for their beds. In other embodiments, bed inventory tracker 206 in central server 200 dynamically generates prices for beds based on the properties of the bed and the respective parameters associated with the facility as discussed above.

In 604, central server 200 next filters bed profiles associated with beds located in the candidate facilities by determining which bed profiles match the criteria specified in the bed. availability request and within the inmate profile of the identified inmate. For example, central server 200 determines bed profiles matching the dates specified in the bed availability request, priced within the price limits specified in the bed availability request, and having neighboring inmates that are compatible with the inmate profile. For example, central server 200 eliminates any beds that have neighboring inmates that have the same gang affiliation as the identified inmate.

In 605, after identifying bed profiles that match the criteria of the bed availability request and the inmate profile, central server 200 generates a list of the bed candidates based on the identified bed profiles. In 606, central server 200 determines whether any bed candidates require manual review or approval by an administrator. For example, some inmates or facilities may require additional attention to ensure security of the facilities and the public. In some embodiments, such inmates or facilities are be tagged or marked by central server 200 and any bed candidates are required to be approved by an administrator prior to sending to the requesting correction agency. If yes, in 607, the bed candidates that require approval or review are transmitted to the appropriate administrator. In 608, central server 200 transmits the bed candidates to the requesting correction agency.

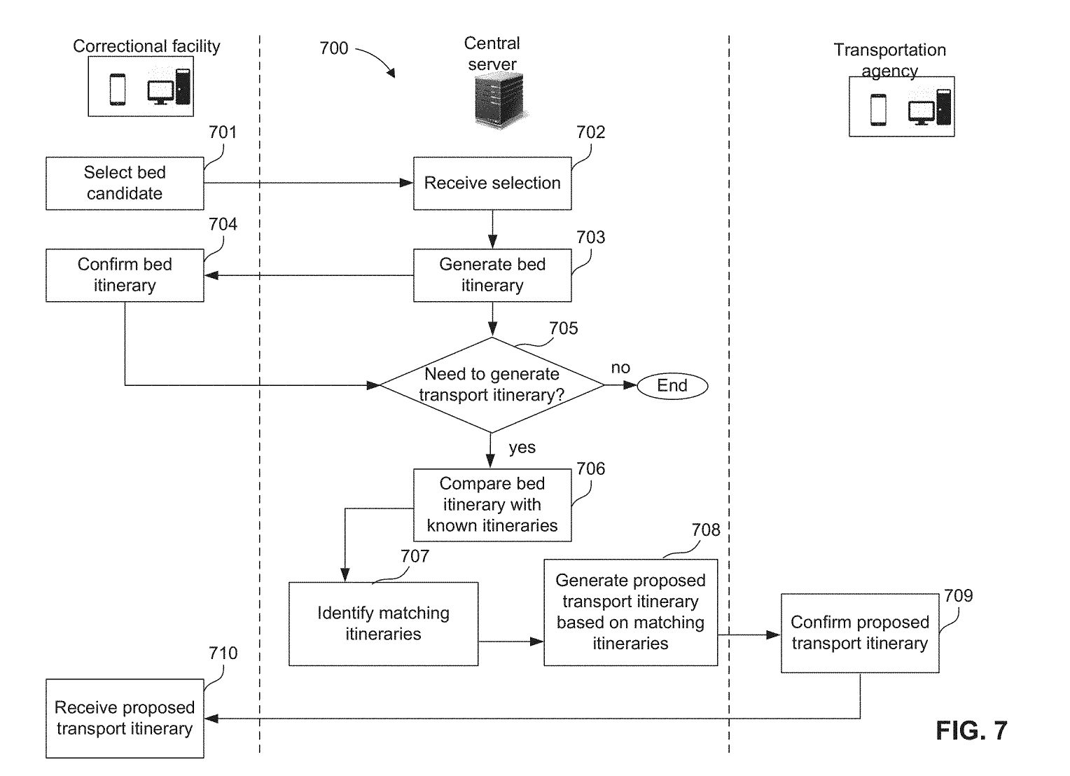

FIG. 7 illustrates a flowchart diagram of an exemplary method 700 for generating a transport itinerary between correction agency 130 and transportation agency 140 within the exemplary inmate bed location system of FIG. 1, according to embodiments of the present disclosure. Method 700 can be performed by processing logic that can comprise hardware (e.g., circuitry, dedicated logic, programmable logic, microcode, etc.), software (e.g., instructions executing on a processing device), or a combination thereof. It is to be appreciated that additional steps, such as additional object recognition steps, may be performed. Moreover, not all steps may be needed to perform the disclosure provided herein. Further, some of the steps may be performed simultaneously, or in a different order than shown in FIG. 7, as will be understood by a person of ordinary skill in the art. Some steps can be combined with steps of methods from other figures and/or performed as a single step. Method 700 can be performed by correction agency 130 central server 200, and transportation agency.

In 701, correction agency 130 selects a bed candidate from the filtered list of bed candidates previously received from central server 200. In 702, central server 200 receives the selection. In some embodiments, central server 200 sends a notification to correction agency 130 and the correctional facility in which the selected bed candidate is located. In 703, central server 200 generates a bed itinerary based on the selected bed candidate. The bed. itinerary includes details of the bed reservation which are derived from criteria in the bed availability request from correction agency 130, the inmate profile, and the bed profile. In some embodiments, the bed itinerary includes at least a bed identifier associated with the selected bed candidate, an identifier associated with the facility in which the bed is located, an inmate identifier associated with the inmate identified in the request from the correction agency, a time period for the reservation which, in some embodiments, are represented by a check-in date and check-out date, in which the bed is reserved for the inmate. Central server 200 then transmits the generated bed itinerary to correction agency 130.

In 704, correction agency 130 confirms the bed itinerary and sends an acknowledgement to the central server 200. In 705, central server 200 determines whether to proceed with generating a transport itinerary associated with the bed itinerary. In some embodiments, central server 200 makes this determination based on a request from correction agency 130, a request from transportation agency 140, or automatically based on preferences associated with the correction agency 130 or central server 200. The transport itinerary includes at least a route for transporting an inmate to the selected correctional facility, an inmate identifier associated with the inmate being transported, and a facility identifier identifying the selected correctional facility. In some embodiments, the transport itinerary includes transport details regarding more than one inmate. Such transport itineraries are generated for inmates that have the same or similar transport parameters such as, for example, being transported to the same correctional facility, being transported to nearby (e.g., within 5 miles) correctional facilities, being transported from the same correction agency, or being transported along the same route (e.g., a correctional facility of one inmate may be on the same route to transport a second inmate to another correctional facility). If not, the process ends.

If central server 200 determines to generate a transport itinerary, in 706, central server 200 compares the details of generated bed itinerary with previously generated bed and transport itineraries. Central server 200 searches for shared details such as the same correctional facility, the same transport route, and the same correction agency to determine whether any previously generated bed and transport itineraries could be matched with the generated bed itinerary. As discussed above, matches between the generated bed itinerary and previously generated bed and transport itineraries are determined based on a number of parameters such as the correctional facility, inmate characteristics (e.g., violence level, security level required by the inmate, gang affiliations), correction agency, and transport routes. In some embodiments, the violence level and security level of an inmate is associated with a security requirements of the correctional facility. For example, the higher the security and/or violence level of an inmate, central server 200 requires higher security for the correctional facility. In 707, central server 200 identifies matching bed and transport itineraries based on any matching parameters. Based on the identified matching bed and transport itineraries, central server 200 further identifies the inmates (e.g., based on associated inmate identifiers), correctional facilities (e.g., based on associated facility identifiers), and transport routes within the matching bed and transport itineraries.

In 708, central server 200 generates a proposed transport itinerary for the generated bed itinerary discussed in 703 based on the parameters identified within the matching bed and transport itineraries. The proposed transport itinerary includes transport details for transporting the inmate to correctional facility 110, such as the transport time, transport route or an identifier of transportation agency 140 that is responsible for transporting the inmate. If there are no matches to any previously generated bed or transport itineraries, the proposed transport itinerary only includes information regarding the inmate identified in the bed availability request from correction agency 130. In other words, the transport details for the inmate are not shared with any other inmates. If there are matches to any previously generated bed or transport itineraries, the proposed transport itinerary will include transport details of the other inmates. In some embodiments, central server 200generates more than one transport itinerary based on the number of matching bed or transport itineraries. For example, if central server 200 determines that there are at least two transportation agencies that are transporting other inmates along similar routes, central server 200 generates two proposed transport itineraries a first proposed transport itinerary for transporting the inmate using the first transportation agency and a second proposed transport itinerary using the second transportation agency.

In 709, central server 200 transmits the proposed transport itinerary to transportation agency 140 which confirms the details of the transport itinerary. In some embodiments, transportation agency 140 can accept the transport itinerary by sending an acknowledgement to central server 200. In some embodiments, transportation agency 140 also modifies details of the transport itinerary including pricing for the transport and the transport route prior to confirming the proposed transport itinerary. In some embodiments, transportation agency 140 can also decline the transport itinerary. If transportation agency confirms the proposed transport itinerary, central server 200 transmits the proposed transport itinerary to correction agency 130 for review and/or acceptance. At this point, correction agency 130 and transportation agency 140 can negotiate details of the transport including pricing, dates, and routes associated with transporting the inmate.

### Exemplary Computer Implementation

It will be apparent to persons skilled in the relevant art(s) that various elements and features of the present disclosure, as described herein, can be implemented in hardware using analog and/or digital circuits, in software, through the execution of computer instructions by one or more general purpose or special-purpose processors, or as a combination of hardware and software.

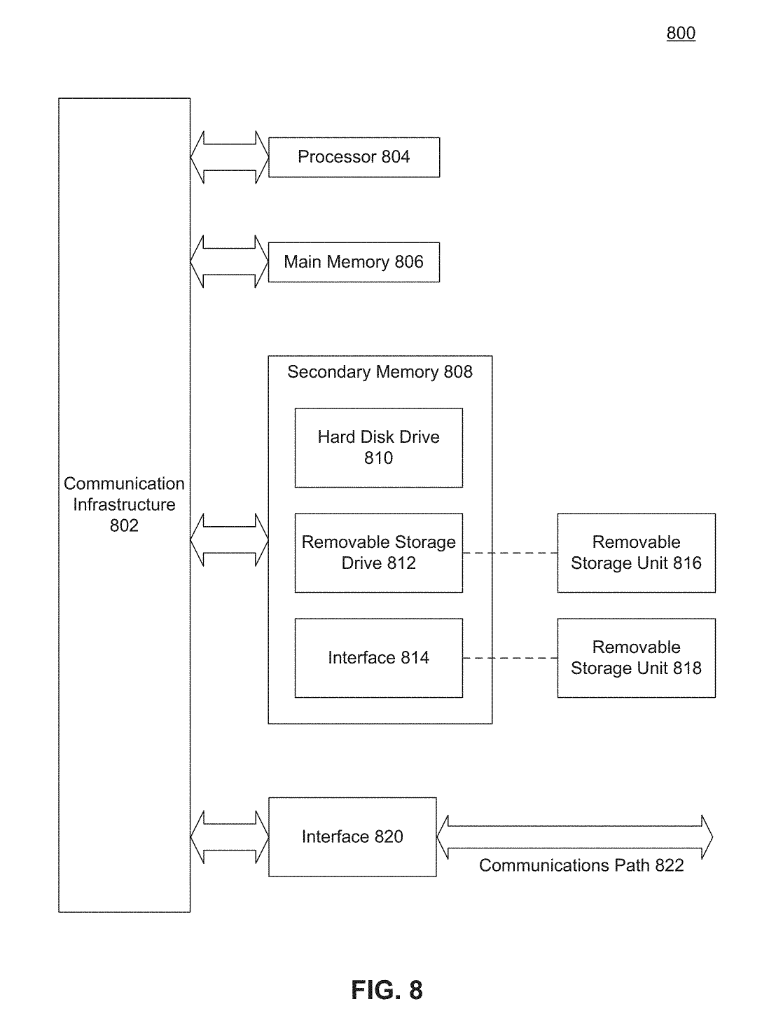

The following description of a general purpose computer system is provided for the sake of completeness. Embodiments of the present disclosure can be implemented in hardware, or as a combination of software and hardware. Consequently, embodiments of the disclosure may be implemented in the environment of a computer system or other processing system. For example, the methods of FIGS. 4-7 can be implemented in the environment of one or more computer systems or other processing systems. An example of such a computer system 800 is shown in FIG. 8. One or more of the modules depicted in the previous figures can be at least partially implemented on one or more distinct computer systems 800.

Computer system 800 includes one or more processors, such as processor 804. Processor 804 can be a special purpose or a general purpose digital signal processor, Processor 804 is connected to a communication infrastructure 802 (for example, a bus or network). Various software implementations are described in terms of this exemplary computer system. After reading this description, it will become apparent to a person skilled in the relevant art(s) how to implement the disclosure using other computer systems and/or computer architectures.

Computer system 800 also includes a main memory 806, preferably random access memory (RAM), and may also include a secondary memory 808. Secondary memory 808 may include, for example, a hard disk drive 810 and/or a removable storage drive 812, representing a floppy disk drive, a magnetic tape drive, an optical disk drive, or the like Removable storage drive 812 reads from and/or writes to a removable storage unit 816 in a well-known manner. Removable storage unit 816 represents a floppy disk, magnetic tape, optical disk, or the like, which is read by and written to by removable storage drive 812. As will be appreciated by persons skilled in the relevant art(s), removable storage unit 816 includes a computer usable storage medium having stored therein computer software and/or data.

In alternative implementations, secondary memory 808 may include other similar means for allowing computer programs or other instructions to be loaded into computer system 800. Such means may include, for example, a removable storage unit 818 and an interface 814. Examples of such means may include a program cartridge and cartridge interface (such as that found in video game devices), a removable memory chip (such as an EPROM, or PROM) and associated socket, a thumb drive and USB port, and other removable storage units 818 and interfaces 814 which allow software and data to be transferred from removable storage unit 818 to computer system 800.

Computer system 800 may also include a communications interface 820.

Communications interface 820 allows software and data to be transferred between computer system 800 and external devices. Examples of communications interface 820 may include a modem, a network interface (such as an Ethernet card), a communications port, a PCMCIA slot and card, etc. Software and data transferred via communications interface 820 are in the form of signals which may be electronic, electromagnetic, optical, or other signals capable of being received by communications interface 820. These signals are provided to communications interface 820 via a communications path 822. Communications path 822 carries signals and may be implemented using wire or cable, fiber optics, a phone line, a cellular phone link, an RF link and other communications channels.

As used herein, the terms “computer program medium” and “computer readable medium” are used to generally refer to tangible storage media such as removable storage units 816 and 818 or a hard disk installed in hard disk drive 810. These computer program products are means for providing software to computer system 800.

Computer programs (also called computer control logic) are stored in main memory 806 and/or secondary memory 808. Computer programs may also be received via communications interface 820. Such computer programs, when executed, enable the computer system 800 to implement the present disclosure as discussed herein. In particular, the computer programs, when executed, enable processor 804 to implement the processes of the present disclosure, such as any of the methods described herein. Accordingly, such computer programs represent controllers of the computer system 800. Where the disclosure is implemented using software, the software may be stored in a computer program product and loaded into computer system 800 using removable storage drive 812, interface 814, or communications interface 820.

In another embodiment, features of the disclosure are implemented primarily in hardware using, for example, hardware components such as application-specific integrated circuits (ASICs) and gate arrays. Implementation of a hardware state machine so as to perform the functions described herein will also be apparent to persons skilled in the relevant art(s).

### Conclusion

It is to be appreciated that the Detailed Description section, and not the Abstract section, is intended to be used to interpret the claims. The Abstract section may set forth one or more, but not all exemplary embodiments, and thus, is not intended to limit the disclosure and the appended claims in any way.

The disclosure has been described above with the aid of functional building blocks illustrating the implementation of specified functions and relationships thereof. The boundaries of these functional building blocks have been arbitrarily defined herein for the convenience of the description. Alternate boundaries may be defined so long as the specified functions and relationships thereof are appropriately performed.

It will be apparent to those skilled in the relevant art(s) that various changes in form and detail can be made therein without departing from the spirit and scope of the disclosure. Thus, the disclosure should not be limited by any of the above-described exemplary embodiments, but should be defined only in accordance with the following claims and their equivalents.

1. A method, comprising:
receiving, from an inventory tracker device associated with a first bed, an inventory update, the inventory update including a bed identifier associated with the bed and a notification regarding an availability of the first bed;
updating an availability schedule based on the inventory update, the availability schedule including the availability of the first bed and a second availability of a second bed, wherein the first bed is associated with a first correctional facility and the second bed is associated with a second correctional facility;
receiving, from an electronic device located remotely from the first and second correctional facilities, a bed request, the bed request including a time period and an inmate identifier associated with an inmate;
retrieving an inmate profile based on the inmate identifier;
comparing the time period to the availability schedule;
determining at least one bed candidate based on the comparing, the at least one bed candidate associated with a bed profile;
generating filtered bed candidates based on comparing inmate information in the inmate profile and bed information in the bed profile; and
providing, to the electronic device, the filtered bed candidates.

2. The method of claim 1, wherein the inventory update further includes a facility identifier, the facility identifier indicating the first correctional facility.

3. The method of claim 2, wherein the inventory tracker device is a tag that is attached to the first bed.

4. The method of claim 1, further comprising:
receiving a bed confirmation selection that includes a selected bed candidate from the filtered bed candidates;
generating a bed itinerary that includes the selected bed candidate, the inmate identifier, and the time period; and
updating the availability schedule based on the bed itinerary.

5. The method of claim 4, further comprising:
retrieving another bed itinerary from a database;
generating a transport itinerary based on comparing the bed itinerary and the another bed itinerary, wherein the transport itinerary includes a transport route for transporting a first inmate associated with the bed itinerary and a second inmate associated with the another bed itinerary; and
transmitting the transport itinerary to a remote device associated with a transportation agency.

6. The method of claim 1, wherein the inmate profile includes a security requirement associated with the inmate, the bed profile includes a security criteria of the first correctional facility, and wherein the comparing inmate information in the inmate profile and bed information in the bed profile further comprises comparing the security requirement and the security criteria.

7. The method of claim 6, wherein the security requirement includes at least one of a security level required for the inmate, a gang affiliation associated with the inmate, a flight risk associated with the inmate, a violence level associated with the inmate.

8. The method of claim 1, wherein the bed profile includes at least one of a facility identifier identifying the first correctional facility in which the first bed is located and a location identifier identifying a cell block of the first correctional facility in which the first bed is located.

9. A server for providing filtered bed candidates, comprising:
a memory configured to store a bed profile and an inmate profile; and
a processor coupled to the memory, the processor configured to:
receive, from an inventory tracker device associated with a first bed, an inventory update, the inventory update including a bed identifier associated with the first bed and a notification regarding an availability of the first bed;
update an availability schedule based on the inventory update, the availability schedule including the availability of the first bed and a second availability of a second bed, wherein the first bed is associated with a first correctional facility and the second bed is associated with a second correctional facility;
receive, from a remote device outside of the first and second correctional facilities, a bed request, the bed request including a time period and an inmate identifier associated with an inmate;
retrieve, from the memory, the inmate profile based on the inmate identifier;
comparing the time period and bed availability in the availability schedule
determine at least one bed candidate based on the comparing, the at least one bed candidate associated with the bed profile;
generate the filtered bed candidates based on comparing inmate information in the inmate profile and bed information in the bed profile; and
provide, to the remote device, the filtered bed candidates.

10. The server of claim 9, wherein the inventory update further includes a facility identifier, the facility identifier indicating the first correctional facility.

11. The server of claim 10, wherein the inventory tracker device is a tag that is attached to the first bed.

12. The server of claim 9, wherein the processor is further configured to:
receive a bed confirmation selection that includes a selected bed candidate from the filtered bed candidates;
generate a bed itinerary that includes the selected bed candidate, the inmate identifier, and the time period; and
update the availability schedule based on the bed itinerary.

13. The server of claim 12, wherein the processor is further configured to:
retrieve another bed itinerary from a database;
generate a transport itinerary based on comparing the bed itinerary and the another bed itinerary, wherein the transport itinerary includes a transport route for transporting a first inmate associated with the bed itinerary and a second inmate associated with the another bed itinerary; and
transmit the transport itinerary to a remote device associated with a transportation agency.

14. The server of claim 9, wherein the inmate profile includes a security requirement associated with the inmate, the bed profile includes a security criteria of the first correctional facility, and wherein the comparing inmate information in the inmate profile and bed information in the bed profile further comprises comparing the security requirement and the security criteria.

15. The server of claim 14, wherein the security requirement includes at least one of a security level required for the inmate, a gang affiliation associated with the inmate, a flight risk associated with the inmate, a violence level associated with the inmate.

16. The server of claim 9, wherein the bed profile includes at least one of a facility identifier identifying the first correctional facility and a location identifier identifying a cell block of the first correctional facility.

17. A method for providing bed availability within a correctional facility, comprising:
receiving, from an inventory tracker device associated with a first bed and the correctional facility, an inventory update message associated with the first bed, wherein the inventory update message comprises an inmate identifier and a bed identifier associated with the first bed;
detecting, based on the inventory update message, an availability of the first bed within the correctional facility;
generating a second inventory update message based on the detecting the availability of the bed, the second inventory update message including at least one of the bed identifier associated with the first bed, the inmate identifier, and a notification regarding the availability of the first bed;
transmitting the second inventory update message to a server, wherein the second inventory update message is configured to cause the server to update an availability schedule based on the second inventory update message, the availability schedule including the availability of the first bed and a second availability of a second bed, wherein the bed is associated with a correctional facility and the second bed is associated with a second correctional facility.

18. The method of claim 17, further comprising:
in response to failing to receive, from the inventory tracker, the inmate identifier associated with the inmate, updating the notification regarding the availability of the bed.

19. The method of claim 17, further comprising detecting a predetermined time for an inventory tracker associated with the bed to transmit the inventory update message to the server.

20. The method of claim 17, further comprising detecting a mobile update parameter that indicates that the inventory update is generated by a mobile device separate from the bed.
